(19) United States
(12) Patent Application Publication
(10) Pub. No.: US 2026/0053968 A1
(43) Pub. Date: Feb. 26, 2026

(54) MODULAR OIL-CONTROLLED INCENSE DIFFUSER

(71) Applicant: MAJESTIC-M&A INTERNATIONAL CO., LTD., TAIPEI (TW)

(72) Inventor: HSUAN-YU CHAO, TAIPEI (TW)

(21) Appl. No.: 18/811,356

(22) Filed: Aug. 21, 2024

(51) Int. Cl. A61L 9/14

(52) U.S. Cl. CPC A61L 9/14; A61L 2209/111; A61L 2209/133; A61L 2209/134

(57) ABSTRACT

A modular oil-control incense diffuser, comprises: a shell body with an fuel tank preset in the inner space, an atomization piece at the bottom of the oil tank, an universal oil control module with an universal connector at one end thereof for a commercial aromatic container connected each other, the other end thereof is configured with a combined oil control pipe correspondingly to extend into the fuel tank. A spring in the oil outlet pipe contacted an oil needle to reach the atomization piece, so as to provide precise oil control and atomization, the nozzle is connected to the atomizing piece to release the atomizing gas to outside of the shell body, to effectively control the dripping speed of essential oils, exert a regulating effect to well control the flow rate and atomization speed of essential oils, greatly improving the atomization efficiency while avoiding the waste of excessive dripping thereof.

### FIELD OF THE INVENTION

The present invention relates to the technical field of incense essential oil diffusers, and particularly refers to a modular oil-controlled incense diffuser.

### THE PRIOR ARTS

In recent years, the medical and beauty industries have developed rapidly, and humans have paid more and more attention to beauty care and medical care. For example, currently, ultrasonic water oxygen machines, beauty and health care machines, water mist incense humidifiers, essential oil diffusers, etc. have been widely used in beauty salons and ordinary households. It is mainly achieved by using the principle of ultrasonic atomization. Through the high-frequency vibration action of the atomization piece, the essence liquid output from the container is atomized into tiny particles of water mist, which can be diffused into the surrounding environment.

However, the early general ultrasonic aroma diffuser requires the essence in the commercially available container to be packed into a special container before being loaded into the aroma diffuser, which is inconvenient for use. The newer design is a universal essential oil diffuser. A commercially available aromatic container can be screwed directly into the universal spray connector of the diffuser body. There is no need to purchase other items or perform separate packaging operations. It can be easily connected at any time. Use for aromatherapy.

However, the following shortcomings will occur in practical applications: 1. Once the aromatic container is turned upside down, the dripping volume and dripping speed of the essential oil cannot be effectively controlled, and the essential oil will be exhausted quickly and cannot be used for a long time. 2. If too much essential oil is dropped, it will cover the atomizer piece and affect the atomization effect. Even if too much essential oil accumulates on the atomizer piece, the atomization cannot be performed normally. 3. In order to save costs, in some structures, the back of the atomizing piece is usually located directly at the end of the oil supply channel. There are no good oil control and anti-leakage measures between the atomizing piece and the oil supply channel, which can easily cause the essential oil bottle to leakage occurs. When filled with essential oil, leakage will occur directly from the atomizer, which is its main disadvantage.

### SUMMARY OF THE INVENTION

In view of this, the main purpose of the present invention is to provide a modular oil-controlled incense diffuser, which comprises: a shell body, an universal oil control module, a nozzle and a control circuit; wherein

The shell body is shaped with appearance structure, a fuel tank is preset in an inner space of the shell body to accommodate the universal oil control module, and an atomization piece is configured at the bottom of the fuel tank.

The universal oil control module is disposed on a partition to cover assembly a top of the fuel tank, the universal oil control module is configured with a first end and an opposite second end, wherein the universal oil control module comprises an universal connector recessed on the first end in a round cylindrical shape, an interface of the universal connector is configured with an internal screw thread and filled with a sealing ring for docking with a commercially available aromatic container, and a combined oil control pipe is protruding from the second end to correspondingly extend into the oil tank, wherein an oil outlet pipe is configured with an oil needle connected by a spring inside the oil outlet pipe, the oil outlet pipe is configured with a constriction flange, the oil needle has a cross-shaped middle expansion section to form a mutual limiter with the constriction flange, wherein a front end of the oil needle that protrudes from the oil outlet pipe reaches the atomization piece to provide precise oil control and atomization.

The nozzle is connected to the atomizing piece to release the atomizing gas to the outside of the shell body.

The control circuit is configured with a circuit board in the shell body, the control circuit is electrically connected to the atomization piece, the shell body is configured with a power plug for providing external power to internal said circuit board, the shell body is configured with a power switch to control the circuit board as switch action.

Compared with the prior art structure, the advantage of the modular oil-controlled incense diffuser of the present invention is that it can be simply installed and used directedly in various shell body through the universal oil control module. Regardless of the size of the shell body, whether desktop or wall-plugged, the general oil control module can be quickly selected and inserted, which can effectively speed up development and improve commercial production efficiency, meeting the practical needs of diversified and trendy designs. The most important feature of the application is that the universal oil control module can effectively control the dripping speed of essential oils, exerting a regulating effect to well control the flow rate and atomization speed of essential oils, greatly improving the atomization efficiency of essential oils, and at the same time, it can avoid condition that excessive dripping of essential oil is a waste.

### BRIEF DESCRIPTION OF THE DRAWINGS

FIG. 1 is a cross-sectional view of the combination of the desktop product of the present invention.

FIG. 2 is a perspective view of the appearance of the desktop product of the present invention.

FIG. 3 is a three-dimensional exploded view of the desktop product of the present invention.

FIG. 4 is a diagram of the first embodiment of the oil-replaceable needle of the present invention.

FIG. 5 is a diagram of the second embodiment of the replaceable oil needle of the present invention.

FIG. 6 is a combined sectional view of the wall plug-in product of the present invention.

FIG. 7 is a perspective view of the appearance of the wall plug-in product of the present invention.

### DETAILED DESCRIPTION OF PREFERRED EMBODIMENTS

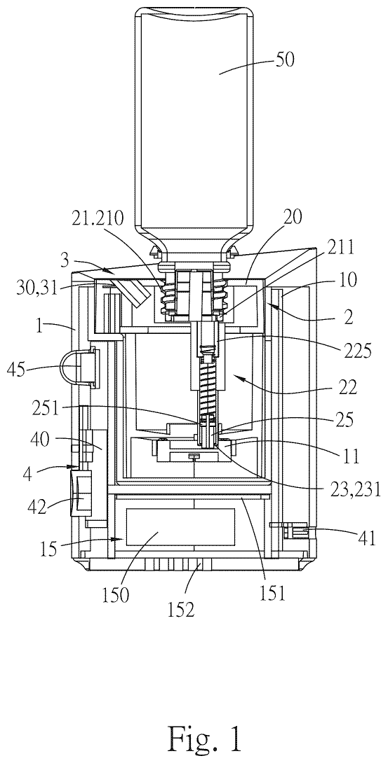

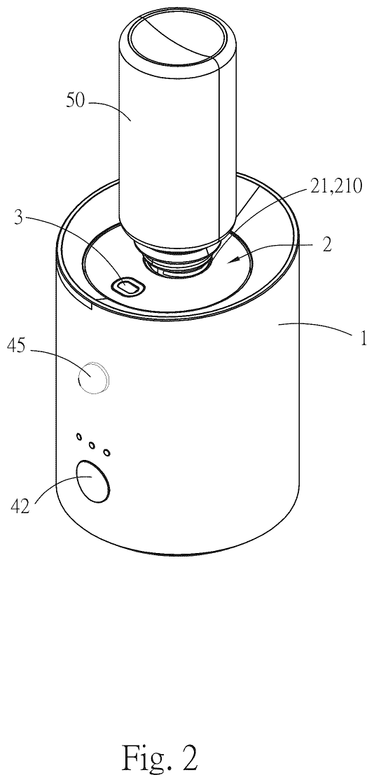

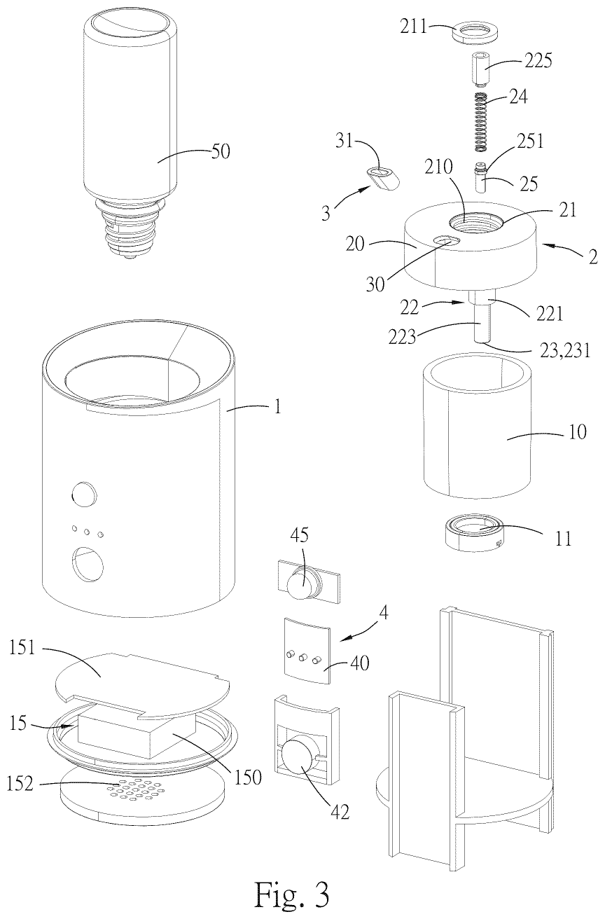

In order to facilitate the understanding of the content of the present invention and the effects that can be achieved, specific embodiments are listed in conjunction with the drawings. The detailed description is as follows: Please refer to FIGS. 1 to 3, the modular oil-controlled incense diffuser of the present invention mainly comprises: a shell body 1, an universal oil control module 2, a nozzle 3 and a control circuit 4; wherein

The shell body 1 is shaped with appearance structure, a fuel tank 10 Is preset in an inner space of the shell body 1 to accommodate the universal oil control module 2, and an atomization piece 11 is configured at the bottom of the fuel tank 10;

One end of the universal oil control module 2 has an universal connector 21 for docking with a commercially available aromatic container 50, and the other end is configured with a combined oil control pipe 22 to extend into the oil tank 10, and the oil needle 25 connected with the spring 24 which is located in the oil outlet pipe 23, the oil needle 25 reaches and contacts the atomization piece 11, so as to provide precise oil control and atomization;

the universal oil control module 2 is disposed on a partition 20 to cover assembly a top of the fuel tank 10, the universal oil control module 2 is configured with a first end and an opposite second end, wherein the universal oil control module 2 comprises an universal connector 21 recessed on the first end in a round cylindrical shape, an interface of the universal connector 21 is configured with an internal screw thread 210 and filled with a sealing ring 211 for docking with a commercially available aromatic container 50, and a combined oil control pipe 22 is protruding from the second end to correspondingly extend into the oil tank 10, wherein an oil outlet pipe 23 is configured with an oil needle 25 connected by a spring 24 inside the oil outlet pipe 23, the oil outlet pipe 23 is configured with a constriction flange 231, the oil needle 25 has a cross-shaped middle expansion section 251 to form a mutual limiter with the constriction flange 231, wherein a front end of the oil needle 25 that protrudes from the oil outlet pipe 23 reaches the atomization piece 11 to provide precise oil control and atomization.

The nozzle 3 is connected and guided to the atomizing piece 11 to release the atomized gas to the outside of the shell body 1. Preferable embodiment, wherein the nozzle 3 is located on the side of the universal connector 21 and consists of a sloped venting tube 30 and a movable nozzle 31, and the sloped venting tube 30 is directly opened on a surface of the hollow partition 20, and;

The control circuit 4 is configured with a circuit board 40 in the shell body 1, the control circuit 4 is electrically connected to the atomization piece 11, the shell body 1 is configured with a power plug 41 for providing external power to internal said circuit board 40, the shell body 1 is configured with a power switch 42 to control the circuit board 40 as switch action.

A preferred embodiment, wherein the partition 20 is in the shape of a hollow sandwich, the combined oil control pipe 22 comprises an outer pipe connected under the universal connector, the outer pipe is composed of an enlargement tube 221 and a reduced pipe 223, the oil needle 25 and the spring 24 are inserted into the reduced pipe 223 from the enlargement tube 221 in sequence, a perforated plug 225 is installed thereon to seal and fix.

Thus, as shown in FIGS. 1 to 3, the modular oil-controlled incense diffuser of the present invention can use the internal screw thread 210 and the sealing ring 211 of the universal connector 21 to be tightly connected to the commercially available aromatic container 50. When docking, when the aromatic container 50 is turned upside down to discharge oil, there is a combined oil control pipe 22 at the other end, and an oil needle 25 connected with a spring 24 is configured in the oil outlet pipe 23 to reach the atomization piece 11. It can effectively control the flow rate and speed of essential oil release, achieving the practical purpose of precise oil atomization control.

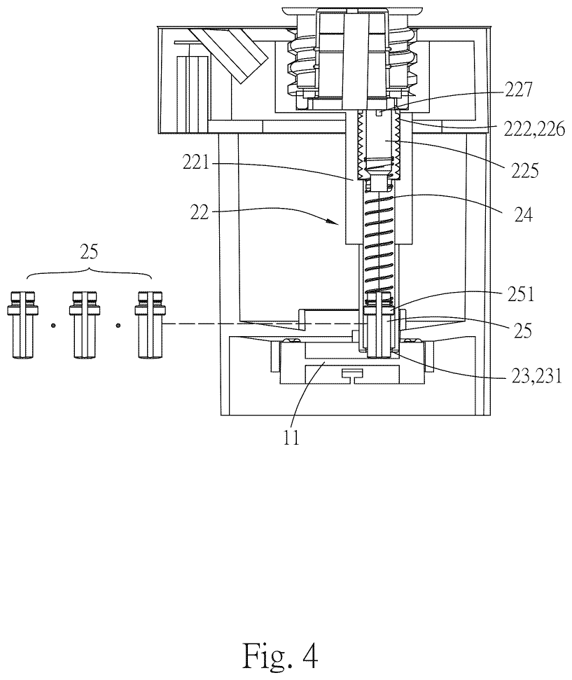

A preferred embodiment, as shown in FIG. 4, in order to meet the concentration of different essential oils to adjust to the optimal dripping speed, the combined oil control pipe 22 can be further configured with said oil needle 25 of different diameters for replacement, and in an opening end of the enlargement tube 221 is configured with another internal screw thread 222, and the perforated plug 225 is correspondingly configured into a screw shape, with an external screw thread 226 on a side of the perforated plug 225 and an opening groove 227 on the top of the perforated plug 225.

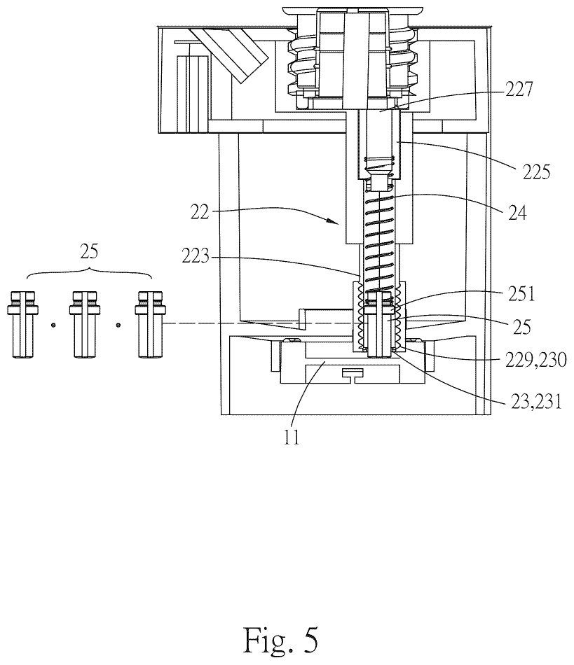

A preferred embodiment is as shown in FIG. 5, wherein the combined oil control pipe 22 is equipped with said oil needle 25 of different diameters for replacement, so as to be suitable for different essential oils and adjust the dripping speed, and a diameter of the reduced pipe 223 is divided into two section, an opening end of the reduced pipe as the first section which is configured with an external screw thread 229, and an opening end of the oil outlet pipe 23 as the second section which is configured in the shape of a nut with the corresponding internal screw thread 230, which can be easily used to unscrew the nut by hand to loosen the oil outlet port 23, take out and replace the appropriate oil needle 25, then assemble and lock in reverse.

A preferable embodiment, the present invention can also be further strengthened in details, wherein the shell body 1 is selected at least one shape of a cylindrical body, a column body, an egg body, an L-shape body, a circle body, a block body, a cube body, a sphere body, an ellipsoid body, a polygonal block and an irregular geometry body, that make it easy to make various molding machines according to the present invention.

A preferable embodiment is as shown in FIGS. 1 to 3, herein a strengthening proliferation device 15 is configured in the shell body 1 below the atomization piece 11, and a fan 150 under a breathable partition 151 is electrically connected to the circuit board 40, an air intake hole 152 is configured under the shell body 1, and a fan 150 is used to enhance the air outlet and improve the spray effect.

A preferable embodiment, the shell body 1 is further configured with an automatic sensing component 45 electrically connected to the circuit board 40, so that the product of the present invention can start spraying through the automatic sensing component 45 without manual pressing.

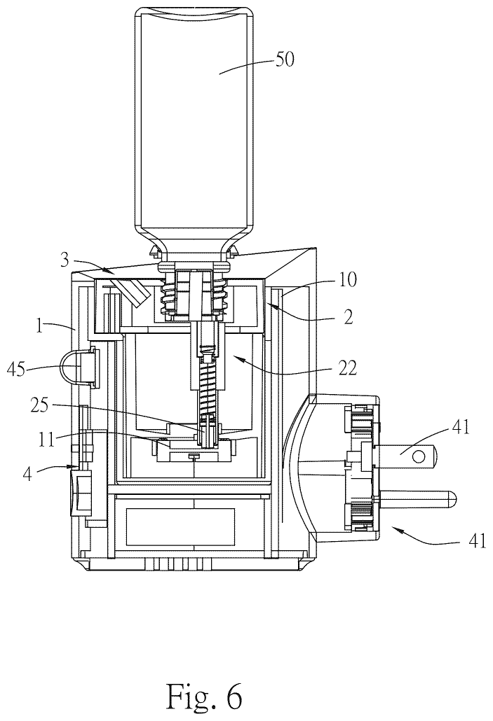

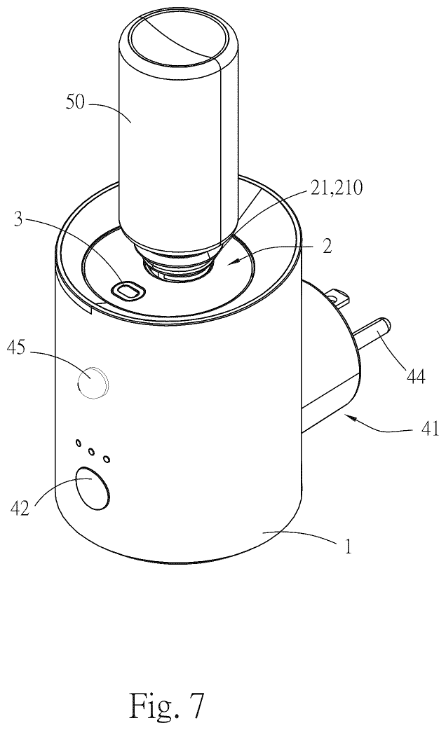

In a preferred implementation, as shown in FIGS. 6 to 7, wherein the shell body 1 is a wall-plug shape, and the power plug 41 is composed of a conjoined rear plug 44, so that the present invention can provide different desktop and wall plug-in products that are available for customers to choose from.

Compared with the prior art structure, the advantage of the modular oil-controlled incense diffuser of the present invention is that it can be simply cooperated and installed in various shell body 1 through the universal oil-control module 2. By using it, no matter whether the multiple sizes of the shell body 1 changes to desktop or wall-plug type, the universal oil control module 2 can be quickly selected and inserted, which can effectively speed up the development speed, improve the efficiency of commercialization production, and meet practical requirements as new trend of diversified shapes for design. The most important feature of the application is that the universal oil control module 2 can effectively control the dripping speed of essential oils, exerting a regulating effect to well control the flow rate and atomization speed of essential oils, greatly improving the atomization efficiency of essential oils, and at the same time, avoid the waste of excessive essential oil dripping.

In summary, the invention is novel and practical and fully meets the patent requirements, and the invention patent application is submitted. However, the foregoing descriptions are only preferred embodiments of the present invention, should not be used to limit the scope of the present invention; therefore, all equivalent changes and modifications made based on the patent scope of the present invention and the content of the creation specification shall be It is within the scope covered by the patent of this invention.

What is claimed is:

1. A modular oil-controlled incense diffuser, wherein comprises: a shell body, an universal oil control module, a nozzle and a control circuit, wherein
the shell body is shaped with appearance structure, a fuel tank is preset in an inner space of the shell body to accommodate the universal oil control module, and an atomization piece is configured at the bottom of the fuel tank;
the universal oil control module is disposed on a partition to cover assembly a top of the fuel tank, the universal oil control module is configured with a first end and an opposite second end, wherein the universal oil control module comprises an universal connector recessed on the first end in a round cylindrical shape, an interface of the universal connector is configured with an internal screw thread and filled with a sealing ring for docking with a commercially available aromatic container, and a combined oil control pipe is protruding from the second end to correspondingly extend into the oil tank, wherein an oil outlet pipe is configured with an oil needle connected by a spring inside the oil outlet pipe, the oil outlet pipe is configured with a constriction flange, the oil needle has a cross-shaped middle expansion section to form a mutual limiter with the constriction flange, wherein a front end of the oil needle that protrudes from the oil outlet pipe reaches the atomization piece to provide precise oil control and atomization;
the nozzle is connected to the atomizing piece to release the atomizing gas to the outside of the shell body; and
the control circuit is configured with a circuit board in the shell body, the control circuit is electrically connected to the atomization piece, the shell body is configured with a power plug for providing external power to internal said circuit board, the shell body is configured with a power switch to control the circuit board as switch action.

2. The modular oil-control incense diffuser as described in claim 1, wherein the partition is in the shape of a hollow sandwich, the combined oil control pipe comprises an outer pipe connected under the universal connector, the outer pipe is composed of an enlargement tube and a reduced pipe, the oil needle and the spring are inserted into the reduced pipe from the enlargement tube in sequence, a perforated plug is installed thereon to seal and fix.

3. The modular oil-controlled incense diffuser as described in claim 2, wherein the nozzle is located on the side of the universal connector and consists of a sloped venting tube and a movable nozzle, and the sloped venting tube is directly opened on a surface of the hollow partition.

4. The modular oil-control incense diffuser as described in claim 2, wherein the combined oil control pipe is equipped with said oil needle of different diameters for replacement, so as to be suitable for different essential oils and adjust the dripping speed, and in an opening end of the enlargement tube is configured with another internal screw thread, and the perforated plug is correspondingly configured into a screw shape, with an external screw thread on a side of the perforated plug and an opening groove on the top of the perforated plug.

5. The modular oil-control incense diffuser described in claim 2, wherein the combined oil control pipe is equipped with said oil needle of different diameters for replacement, so as to be suitable for different essential oils and adjust the dripping speed, and a diameter of the reduced pipe is divided into two section, an opening end of the reduced pipe as the first section which is configured with an external screw thread, and an opening end of the oil outlet pipe as the second section which is configured in the shape of a nut with the corresponding internal screw thread.

6. The modular oil-controlled incense diffuser as described in claim 2, wherein a strengthening proliferation device is configured in the shell body below the atomization piece, and a fan under a breathable partition is electrically connected to the circuit board, an air intake hole is configured under the shell body.

7. The modular oil-controlled incense diffuser as described in claim 2, wherein the shell body is selected at least one shape of a cylindrical body, a column body, an egg body, an L-shape body, a circle body, a block body, a cube body, a sphere body, an ellipsoid body, a polygonal block and an irregular geometry body.

8. The modular oil-controlled incense diffuser as described in claim 2, wherein the shell body is configured with an automatic sensing component electrically connected to the circuit board.

9. The modular oil-controlled incense diffuser as described in claim 8, wherein the shell body is a wall-plug shape, and the power plug is composed of a conjoined rear plug.
